(19) United States
(12) Patent Application Publication
(10) Pub. No.: US 2017/0296043 A1
(43) Pub. Date: Oct. 19, 2017

(54) ENDOSCOPE SYSTEM, ENDOSCOPE APPARATUS, AND METHOD FOR CONTROLLING ENDOSCOPE SYSTEM

(71) Applicant: OLYMPUS CORPORATION, Tokyo (JP)

(72) Inventor: Seigo ON, Tokyo (JP)

(73) Assignee: OLYMPUS CORPORATION, Tokyo (JP)

(21) Appl. No.: 15/637,235

(22) Filed: Jun. 29, 2017

Related U.S. Application Data
(63) Continuation of application No. PCT/JP2015/050434, filed on Jan. 9, 2015.

(51) Int. Cl. A61B 1/04; A61B 1/00

(52) U.S. Cl. CPC A61B 1/041; A61B 1/04; A61B 1/00013; A61B 1/00039; A61B 1/00048

(57) ABSTRACT

An endoscope system includes a capsule endoscope that includes an imaging section, a first processing section that causes the imaging section to operate in a first mode or a second mode, and a first communication section that transmits the captured images to an external device, and the external device that includes a second processing section that outputs a mode switch instruction based on the captured images, and a second communication section that transmits the mode switch instruction, wherein the first processing section causes the imaging section to operate in the second mode from a halfway position of the small intestine, and also operate in the second mode in the large intestine based on the mode switch instruction.

### CROSS REFERENCE TO RELATED APPLICATION

This application is a continuation of International Patent Application No. PCT/JP2015/050434, having an international filing date of Jan. 9, 2015, which designated the United States, the entirety of which is incorporated herein by reference.

### BACKGROUND

The present invention relates to an endoscope system, an endoscope apparatus, a method for controlling an endoscope system, and the like.

In recent years, a capsule-type endoscope apparatus (capsule endoscope) that includes a small imaging section has become widely known. Since the capsule endoscope has a small size, the capsule endoscope is designed so that the frame rate is controlled to reduce the number of captured images from the viewpoint of a reduction in power consumption and the like. The frame rate is controlled corresponding to the speed at which the capsule endoscope moves within the digestive tract, for example. More specifically, the frame rate is decreased when the capsule endoscope moves at a low speed, and is increased when the capsule endoscope moves at a high speed.

JP-A-2006-223892 discloses a method that analyzes the motion of the capsule using an image captured by the capsule main body that has been swallowed, and adaptively controls the capture frame rate. Specifically, the capture frame rate is decreased when the motion of the capsule is relatively slow, and is increased when the motion of the capsule is relatively fast.

### SUMMARY

According to one aspect of the invention, there is provided an endoscope system comprising:

a capsule endoscope; and

an external device,

the capsule endoscope comprising:

an imaging section that captures a small intestine and a large intestine to acquire a plurality of captured images in time series;

a first processor that comprises hardware, and controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate; and

a first communication section that transmits the captured images to the external device, and

the external device comprising:

a second processor that comprises hardware, and outputs a mode switch instruction based on the captured images, the mode switch instruction instructing to switch from the first mode to the second mode at a halfway position of the small intestine; and

a second communication section that transmits the mode switch instruction to the first communication section,

wherein the first processor switches the imaging section from the first mode to the second mode at the halfway position of the small intestine based on the mode switch instruction, and causes the imaging section to operate in the second mode from the halfway position of the small intestine, and also operate in the second mode in the large intestine.

According to another aspect of the invention, there is provided an endoscope apparatus comprising:

an imaging section that captures a small intestine and a large intestine to acquire a plurality of captured images in time series; and

a processor that comprises hardware, and controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate,

the endoscope apparatus switching the imaging section from the first mode to the second mode at a halfway position of the small intestine based on the captured images, and causing the imaging section to operate in the second mode from the halfway position of the small intestine, and also operate in the second mode in the large intestine.

According to another aspect of the invention, there is provided a method for controlling an endoscope system comprising:

causing an imaging section to capture a small intestine and a large intestine to acquire a plurality of captured images in time series;

outputting a mode switch instruction based on the captured images, the mode switch instruction instructing to switch from a first mode to a second mode at a halfway position of the small intestine, the first mode being a mode in which the imaging section captures an image at a first frame rate, and the second mode being a mode in which the imaging section captures an image at a second frame rate that is at least higher than the first frame rate; and

switching the imaging section from the first mode to the second mode at the halfway position of the small intestine based on the mode switch instruction, and causing the imaging section to operate in the second mode from the halfway position of the small intestine, and also operate in the second anode in the large intestine.

### BRIEF DESCRIPTION OF THE DRAWINGS

FIG. 1 illustrates a configuration example of an endoscope system according to the embodiments of the invention.

FIG. 2 illustrates a detailed configuration example of an endoscope system according to one embodiment of the invention.

FIG. 3 illustrates a configuration example of an endoscope apparatus (capsule endoscope) according to one embodiment of the invention.

FIG. 4 is a flowchart illustrating a process according to one embodiment of the invention.

FIG. 5 illustrates a configuration example of a switch determination section.

FIG. 6 is a view illustrating an area setting process and a local feature quantity calculation process.

FIG. 7 is a view illustrating an LBP feature quantity calculation process.

FIG. 8 is a view illustrating an HSV feature quantity calculation process.

FIG. 9 is a view illustrating an HOB feature quantity calculation process.

FIG. 10 is a view illustrating a color-related local feature quantity.

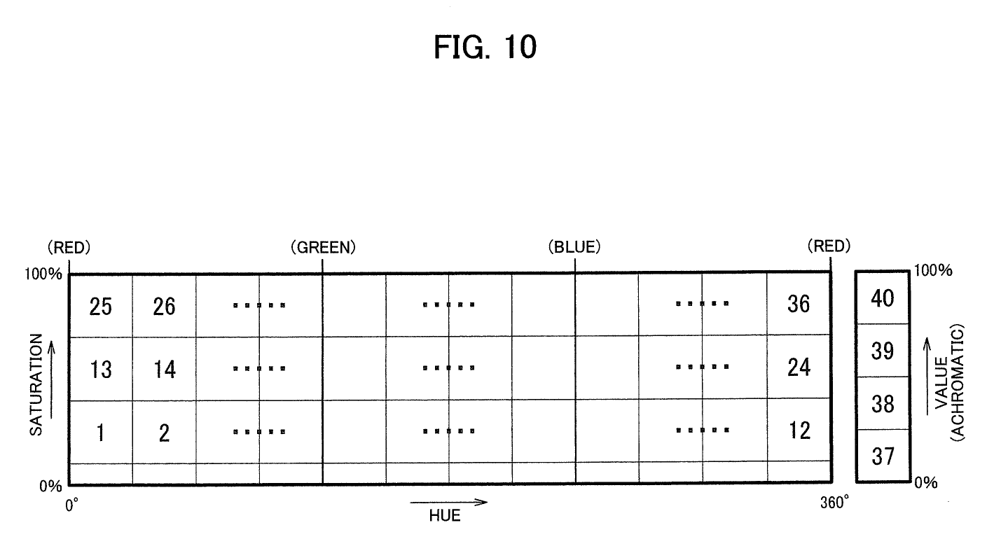

FIG. 11 is a view illustrating a method that sets an interval, and detects a halfway position of the small intestine.

FIG. 12 is a view illustrating the flow of a BoF algorithm process.

FIG. 13 is a view illustrating an individual variation in villus distribution (i.e., the villus distribution of each user).

### DESCRIPTION OF EXEMPLARY EMBODIMENTS

According to one embodiment of the invention, there is provided an endoscope system comprising:

a capsule endoscope; and

an external device,

the capsule endoscope comprising:

an imaging section that captures a small intestine and a large intestine to acquire a plurality of captured images in time series;

a first processor that comprises hardware, and controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate; and

a first communication section that transmits the captured images to the external device, and

the external device comprising:

a second processor that comprises hardware, and outputs a mode switch instruction based on the captured images, the mode switch instruction instructing to switch from the first mode to the second mode at a halfway position of the small intestine; and

a second communication section that transmits the mode switch instruction to the first communication section,

wherein the first processor switches the imaging section from the first mode to the second mode at the halfway position of the small intestine based on the mode switch instruction, and causes the imaging section to operate in the second mode from the halfway position of the small intestine, and also operate in the second mode in the large intestine.

According to another embodiment of the invention, there is provided an endoscope apparatus comprising:

an imaging section that captures a small intestine and a large intestine to acquire a plurality of captured images in time series; and

a processor that comprises hardware, and controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate,

the endoscope apparatus switching the imaging section from the first mode to the second mode at a halfway position of the small intestine based on the captured images, and causing the imaging section to operate in the second mode from the halfway position of the small intestine, and also operate in the second mode in the large intestine.

According to another embodiment of the invention, there is provided a method for controlling an endoscope system comprising:

causing an imaging section to capture a small intestine and a large intestine to acquire a plurality of captured images in time series;

outputting a mode switch instruction based on the captured images, the mode switch instruction instructing to switch from a first mode to a second mode at a halfway position of the small intestine, the first mode being a mode in which the imaging section captures an image at a first frame rate, and the second mode being a mode in which the imaging section captures an image at a second frame rate that is at least higher than the first frame rate; and

switching the imaging section from the first mode to the second mode at the halfway position of the small intestine based on the mode switch instruction, and causing the imaging section to operate in the second mode from the halfway position of the small intestine, and also operate in the second mode in the large intestine.

The exemplary embodiments of the invention are described below. Note that the exemplary embodiments described below do not in any way limit the scope of the invention laid out in the claims. Note also that all of the elements described below in connection with the exemplary embodiments should not necessarily be taken as essential elements of the invention.

### 1. Method

A method used in connection with the exemplary embodiments of the invention is described below. A capsule endoscope is limited in battery capacity since it is necessary to reduce the size of the main body. It is ideal to necessarily capture an image at a sufficient frame rate until the capsule endoscope that has been swallowed by the user is discharged from the body. However, it is difficult to meet such a requirement at present due to a limitation in battery capacity. A method that changes the frame rate of a capsule endoscope is widely known. For example, JP-A-2006-223892 discloses a method that controls the frame rate of a capsule endoscope based on the motion of the capsule endoscope.

However, the method disclosed in JP-A-2006-223892 does not take account of whether or not the object that is being captured is an object that should be captured (e.g., specific part). For example, a capsule endoscope according to the exemplary embodiments of the invention is mainly used to observe the large intestine. In this case, if the motion speed of the capsule endoscope has increased for some reason within the stomach or the small intestine, the stomach or the small intestine is captured at a high frame rate, and the battery charge may be insufficient when the capsule endoscope has reached the large intestine. Since the frame rate is not increased even when the capsule endoscope is moving within the large intestine unless the motion speed of the capsule endoscope increases, the large intestine may be captured at a low frame rate. In order to ensure that the user (e.g., doctor) can make an accurate diagnosis, a situation in which the object of interest is missed should be prevented as much as possible, and it is highly desirable to capture the object of interest at a high frame rate.

It is possible to deal with the above problem when it is possible to detect the current position of the capsule endoscope (i.e., the object that is being captured), or detect whether or not the capsule endoscope is situated within the part of interest (i.e., whether or not the object of interest is being captured). Specifically, it is possible to efficiently capture the object of interest even when the battery capacity is limited, by capturing the object of interest at a high frame rate, and capturing an object (object of no interest) other than the object of interest at a low frame rate.

For example, when the object of interest is the large intestine, the start position of the large intestine (i.e., the end point of the large intestine that is situated on the side of the small intestine (i.e., the boundary between the small intestine and the large intestine) may be detected by performing image processing on the captured image. However, since the start position of the large intestine does not have a significant feature within an image, it is difficult to determine the start position of the large intestine by performing image processing on the captured image. A residue is often captured within a digestive organ (e.g., large intestine), and the structure (e.g., wall surface) of the digestive tract may be hidden behind the residue, whereby the detection process by means of image processing may be hindered.

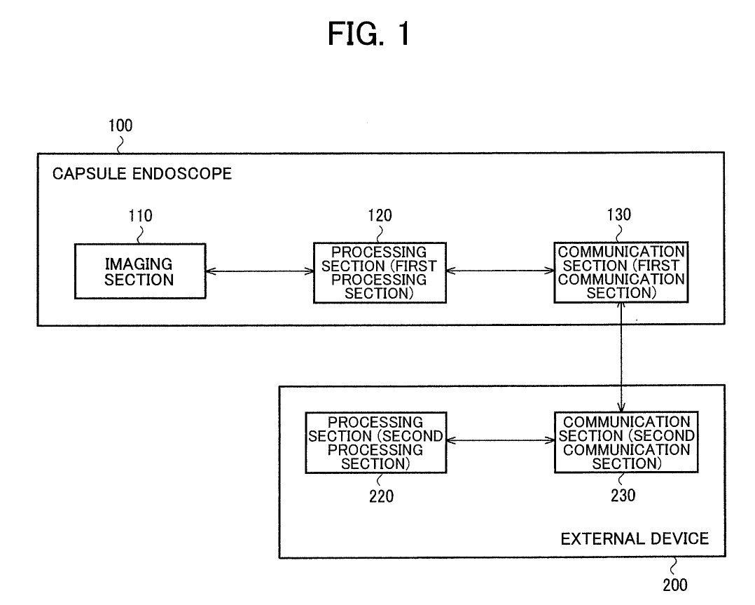

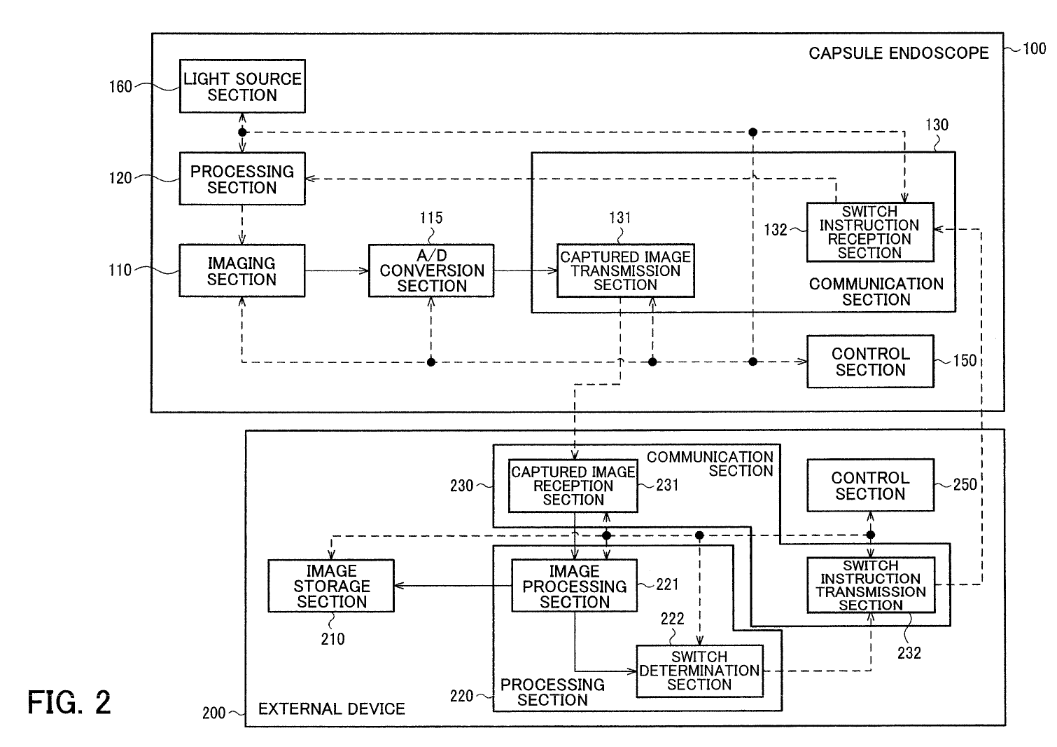

When image processing is performed by an external device other than the capsule endoscope, it is necessary for the capsule endoscope to perform a process that transmits the captured image to the external device, and a process that receives the detection result (mode switch instruction described later in a narrow sense) from the external device (as described later with reference to FIGS. 1 and 2). Therefore, a delay due to the transmission process and the reception process occurs until the frame rate is switched to a high frame rate after the captured image has been acquired. In this case, even if the start position of the large intestine has been accurately detected, the capsule endoscope may enter the large intestine during the delay time, and an area around the start position of the large intestine may be captured at a low frame rate.

On the other hand, it is relatively easy to detect the start position of the small intestine (i.e., the end point of the small intestine that is situated on the side of the stomach (i.e., the boundary between the stomach and the small intestine)) based on the captured image. Specifically, the small intestine has a characteristic villus structure, and the stomach does not have such a villus structure. Therefore, the start position of the small intestine can be detected by detecting the villus structure (villus distribution) by means of image processing. Specifically, a point at which a state in which the villus distribution is small (i.e., a state in which the villus distribution is not observed in a narrow sense) has changed to a state in which the villus distribution is large may be deter wined to be the start position of the small intestine.

However, when the object of interest is the large intestine, it is not sufficient to merely accurately detect the start position of the small intestine. Specifically, since it is necessary to set the frame rate to a high frame rate from the start position of the small intestine to the discharge point of the capsule endoscope in order to reliably capture the large intestine at a high frame rate, an area of no interest is also captured at a high frame rate. Since it takes several hours on average to capture the small intestine, the battery may become almost empty as a result of capturing the entire small intestine at a high frame rate, and it may be impossible to capture the large intestine at a high frame rate.

In order to solve the above problems, the invention proposes a method that reduces the possibility that the object of interest is captured at a low frame rate, and prevents a situation in which an object of no interest is captured at a high frame rate as much as possible. As illustrated in FIG. 1, an endoscope system according to the exemplary embodiments of the invention includes a capsule endoscope 100 and an external device 200, wherein the capsule endoscope 100 includes an imaging section 110 that captures the small intestine and the large intestine to acquire a plurality of captured images in time series, a processing section (first processing section) 120 that controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate, and a communication section (first communication section) 130 that transmits the captured images to the external device 200, and the external device 200 includes a processing section (second processing section) 220 that outputs a mode switch instruction based on the captured images, the mode switch instruction instructing to switch from the first mode to the second mode at a halfway position of the small intestine, and a communication section (second communication section) 230 that transmits the mode switch instruction to the first communication section 130. The first processing section 120 switches the imaging section 110 from the first mode to the second mode at the halfway position of the small intestine based on the mode switch instruction, and causes the imaging section 110 to operate in the second mode from the halfway position of the small intestine, and also operate in the second mode in the large intestine.

Note that the halfway position of the small intestine refers to a position that is situated on the side of the anus with respect to the start position of the small intestine, and is situated on the side of the mouth with respect to the end position of the small intestine (i.e., the boundary between the small intestine and the large intestine). More specifically, when the total length of the small intestine is referred to as L, the halfway position of the small intestine may be a position included within a range from p×L to q×L with respect to the start position of the small intestine. Note that p and q are numbers that satisfy 0<p≦q<1. Specific values of p and q are not limited. For example, p may be 0.2, and q may be 0.8.

According to the method according to the embodiments of the invention, the small intestine is captured at a high frame rate from an intermediate position of the small intestine, and the large intestine is continuously captured at a high frame rate. Therefore, it is possible to increase the possibility that the large intestine is captured at a high frame rate. It is also possible to reduce an area of the small intestine that is captured at a high frame rate (i.e., reduce the time in which the small intestine is captured at a high frame rate) as compared with the case where the small intestine is captured at a high frame rate from the start position of the small intestine. This makes it possible to reduce the consumption of the battery due to capturing of an object of no interest, and efficiently use the battery charge for capturing the large intestine (i.e., object of interest).

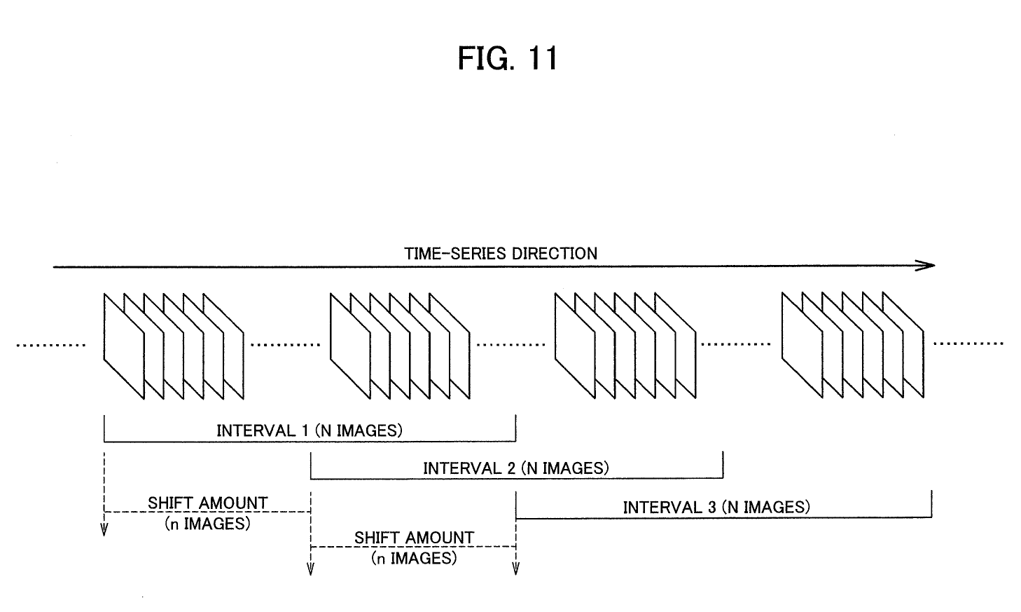

According to the method according to the embodiments of the invention, it is unnecessary to detect the position of the boundary between the small intestine and the large intestine, and it suffices to detect a position that is situated on the side of the large intestine to a certain extent with respect to the start position of the small intestine. Specifically, it is possible to use a certain amount of time and a certain number of captured images when detecting the halfway position of the small intestine. Therefore, a serious problem does not occur even when a delay due to the transmission process and the reception process occurs until the frame rate is switched to a high frame rate after the captured image has been acquired using the configuration illustrated in FIG. 1, for example. Specifically, since it is considered that the capsule endoscope 100 is moving within the small intestine at a timing around the timing at which the halfway position of the small intestine is detected, it is unlikely that the capsule endoscope 100 enters the large intestine until the frame rate is switched to a high frame rate after the captured image has been acquired, and it is unlikely that part of the large intestine is not captured (or the large intestine is captured at a low frame rate). Moreover, since a certain number of captured images can be used for the detection process, it is possible to improve the accuracy of the detection process (as described later with reference to FIG. 11).

First to third embodiments of the invention are described below. A basic processing example will be described in connection with the first embodiment, and a method that detects the halfway position of the small intestine using a learning process will be described in connection with the second embodiment. A method that uses the learning process, and takes account of an individual variation in villus distribution (i.e., the villus distribution of each user) will be described in connection with the third embodiment.

### 2. First Embodiment

FIG. 2 illustrates a configuration example of an endoscope system according to the first embodiment. The endoscope system includes a capsule endoscope 100 and an external device 200. The capsule endoscope 100 includes an imaging section 110 (image sensor), an A/D conversion section 115, a processing section 120 (processor), a communication section 130 (communication circuit and communication interface), a control section 150 (processor), and a light source section 160. The communication section 130 includes a captured image transmission section 131 and a switch instruction reception section 132.

The external device 200 includes an image storage section 210 (memory), a processing section 220 (processor), a communication section 230 (communication circuit and communication interface), and a control section 250 (processor). The processing section 220 includes an image processing section 221 and a switch determination section 222, and the communication section 230 includes a captured image reception section 231 and a switch instruction transmission section 232.

The capsule endoscope 100 is configured so that light that is emitted from the light source section 160 is applied to an object other than the capsule endoscope 100 under control of the control section 150. The reflected light from the object enters the image sensor included in the imaging section 110 through an optical lens system included in the imaging section 110. An analog captured image output from the image sensor included in the imaging section 110 is transmitted to the A/D conversion section 115. The first embodiment utilizes a primary-color single-chip image sensor.

The imaging section 110 is connected to the captured image transmission section 131 through the A/D conversion section 115. The captured image transmission section 131 is connected to the captured image reception section 231 included in the external device 200 through a wireless communication channel. The switch instruction transmission section 232 included in the external device 200 is connected to the switch instruction reception section 132 through a wireless communication channel. The processing section (first processing section) 120 is connected to the imaging section 110. The control section 150 is bidirectionally connected to the imaging section 110, the A/D conversion section 115, the processing section 120, the captured image transmission section 131, the switch instruction reception section 132, and the light source section 160.

The A/D conversion section 115 converts the analog captured image output from the imaging section 110 into a digital captured image (hereinafter referred to as “captured image”), and transmits the captured image to the captured image transmission section 131 under control of the control section 150. The captured image transmission section 131 transmits the captured image to the captured image reception section 231 included in the external device 200 through a wireless communication channel under control of the control section 150.

Although an example in which the captured image is transmitted to the external device 200 through a wireless communication channel without being compressed has been described above, the configuration is not limited thereto. For example, the captured image may be compressed, and then transmitted to the external device 200.

In the first embodiment, the image capture frame rate (hereinafter referred to as “capture FR”) is controlled by a given processing mechanism using a determination control signal (switch instruction and mode switch instruction) output from the switch instruction transmission section 232 included in the external device 200. The process performed by the processing section 120 will be described after describing the process performed by the external device 200.

The external device 200 is configured so that the captured image reception section 231 is connected to the image storage section 210 and the switch determination section 222 through the image processing section 221. The switch determination section 222 is connected to the switch instruction transmission section 232. The switch instruction transmission section 232 is connected to the switch instruction reception section 132 included in the capsule endoscope 100 through a wireless communication channel. The control section 250 is bidirectionally connected to the image storage section 210, the image processing section 221, the switch determination section 222, the captured image reception section 231, and the switch instruction transmission section 232.

The captured image reception section 231 receives the captured image transmitted from the capsule endoscope 100 through a wireless communication channel, and transmits the captured image to the image processing section 221.

The image processing section 221 performs image processing on the captured image transmitted from the captured image reception section 231 under control of the control section 250. For example, the image processing section 221 performs an interpolation process, a color management process, an edge enhancement process, a grayscale transformation process, and the like known in the art. The image processing section 221 transmits the resulting RGB captured image to the image storage section 210 under control of the control section 250, and the RGB captured image is stored in the image storage section 210. The image processing section 221 also transmits the captured image to the switch determination section 222 under control of the control section 250.

The first embodiment utilizes a capsule endoscope that is used to make a diagnosis with respect to the large intestine (see above). In order to prevent a situation in which an erroneous diagnosis is made with respect to the large intestine, and reduce power consumption, it is ideal to capture an image at a low frame rate until the capsule endoscope that has been swallowed by the patient reaches the inlet of the large intestine, and capture an image at a high frame rate after the capsule endoscope has entered the large intestine. However, it is difficult to detect the inlet of the large intestine in real time due to the effects of a residue, bubbles, the motion of the capsule, an individual variation in the structure of the small intestine and the large intestine (i.e., the structure of the small intestine and the large intestine of each patient), and the like. Therefore, there is a risk that the inlet of the large intestine is not detected, and an image is continuously captured at a low frame rate even after the capsule endoscope has entered the large intestine. According to the first embodiment, an image is captured at a low frame rate (e.g., 2 fps) until the capsule endoscope that has been swallowed by the patient reaches a given halfway area of the small intestine, and the frame rate is switched to a high frame rate (e.g., 12 fps) after the capsule endoscope has reached the given halfway area of the small intestine. The first embodiment is characterized by specifying the given halfway area of the small intestine.

The small intestine consists of the duodenum, the jejunum, and the ileum. The stomach is connected to the jejunum through the duodenum, and the ileum is connected to the large intestine (colon) through the ileocecal valve. There is no clear anatomical boundary between the jejunum and the ileum. About ⅖th of the jejunum-ileum area that is situated on the side of the mouth is normally determined to be the jejunum, and the remaining area is normally determined to be the ileum. A villus is a structure specific to the small intestine. Villi are most densely observed in the duodenum. The villus density decreases toward the end of the ileum (toward the large intestine). The villus distribution in the jejunum is denser than the villus distribution in the ileum. In the first embodiment, the villus distribution in the duodenum, the jejunum, and the ileum is taken into consideration, and the villus distribution is determined from the captured image based on the image recognition process. An approximate boundary between the jejunum and the ileum is determined by utilizing identification information about the villus distribution, and used as the given halfway area of the small intestine that is used to switch the capture frame rate from a low frame rate to a high frame rate. Note that it suffices to detect the halfway position of the small intestine (i.e., a position that is situated on the side of the anus with respect to the start position of the small intestine to such an extent that the battery consumption can be reduced, and is situated on the side of the mouth with respect to the end position of the small intestine to such an extent that it is possible to prevent a situation in which part of the large intestine is not captured) (as described below). Specifically, the boundary between specific parts need not necessarily be detected in a strict way.

Specifically, the switch determination section 222 continuously determines the villus distribution in the intestine with respect to images captured in time series, and, when a decrease in villus distribution has been detected, determines the position of the small intestine at which the capsule main body is situated to be the given halfway area of the small intestine. The switch determination section 222 transmits determination information to the switch instruction reception section 132 included in the capsule main body in real time through the switch instruction transmission section 232 and a wireless communication channel under control of the control section 250. The switch instruction reception section 132 transmits the determination information to the processing section 120 under control of the control section 150. The processing section 120 switches the capture mode from a low-frame-rate capture mode to a high-frame-rate capture mode under control of the control section 150.

Although an example in which an image is captured at a low frame rate (e.g., 2 fps) until the capsule endoscope reaches the given halfway area of the small intestine, and the frame rate is switched to a high frame rate (e.g., 8 fps) after the capsule endoscope has reached the given halfway area of the small intestine, has been described above, the configuration is not limited thereto. For example, an image is captured at a low frame rate (e.g., 2 fps) until the capsule endoscope reaches the given halfway area of the small intestine, and the frame rate is switched to a high frame rate (e.g., 8 fps) when it has been determined that the villus distribution in the small intestine has decreased, and switched to a super-high frame rate (e.g., 16 fps) when the villus distribution in the small intestine has further decreased. The frame rate may be switched in a plurality of steps (e.g., three or more steps) corresponding to the villus distribution as described above.

The frame rate need not necessarily be switched from a high frame rate to a super-high frame rate based on the villus distribution. For example, when one low frame rate (e.g., 2 fps) and two high frame rates (e.g., 8 fps and 16 fps) are provided, an image is captured at a low frame rate until the capsule endoscope reaches the given halfway area of the small intestine, and the capture mode is switched to a high-frame-rate mode when it has been determined that the villus distribution in the small intestine has decreased. In this case, the motion of the capsule main body is detected in the high-frame-rate mode, and the capture frame rate is controlled in a plurality of steps. When the motion of the capsule main body is not detected, or when the motion of the capsule main body is small, an image is captured at a high frame rate 1 (e.g., 8 fps). When the motion of the capsule main body is large, an image is captured at a high frame rate 2 (e.g., 16 fps). Specifically, an image is captured using a multi-step capture frame rate corresponding to the motion of the capsule main body after the capsule main body has reached the given halfway area of the small intestine in order to prevent a situation in which an erroneous diagnosis is made. The magnitude of the motion of the capsule main body may be detected using a plurality of images captured in time series, or may be detected using a motion detection sensor or the like.

According to the first embodiment and the modifications thereof in which the villus distribution is detected using the captured image, and an image is captured at a low frame rate until the capsule main body that has been swallowed reaches the given halfway area of the small intestine, and captured one or more high frame rates after the capsule main body has reached the given halfway area of the small intestine until the capsule main body is discharged from the large intestine (body), it is possible to prevent a situation in which an erroneous diagnosis is made with respect to the large intestine, and reduce the power consumption of the capsule endoscope 100.

Although an example in which the image captured by the main body of the capsule endoscope 100 is transmitted to the external device 200, and the external device 200 detects the given halfway area of the small intestine, has been described above, the configuration is not limited thereto. For example, the main body of the capsule endoscope 100 may be provided with a configuration that detects the given halfway area of the small intestine.

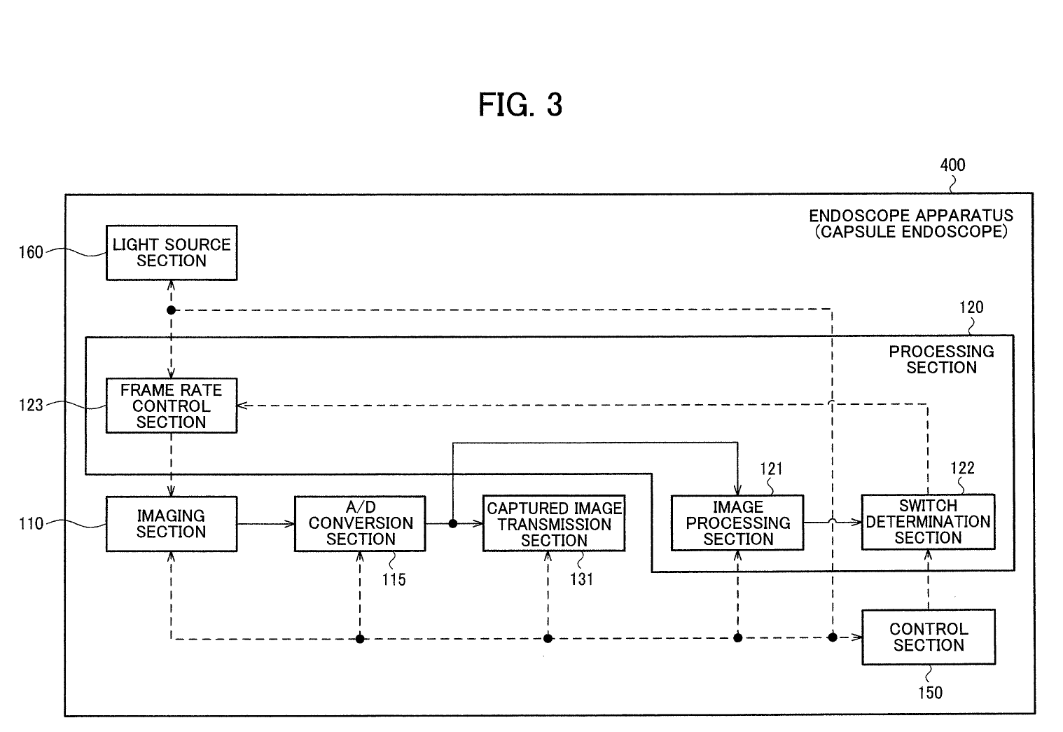

FIG. 3 illustrates a configuration example of an endoscope apparatus (capsule endoscope) 400 that is employed in such a case. As illustrated in FIG. 3, the endoscope apparatus 400 includes an imaging section 110, an A/D conversion section 115, a processing section 120, a captured image transmission section 131, a control section 150, and a light source section 160. The processing section 120 includes an image processing section 121, a switch determination section 122, and a frame rate control section 123.

The imaging section 110, the A/D conversion section 115, the control section 150, and the light source section 160 are the same as those described above with reference to FIG. 2.

The captured image transmission section 131 transmits the captured image to the outside. In the example illustrated in FIG. 3, since the endoscope apparatus performs the process that detects the halfway position of the small intestine based on the captured image, the captured image transmitted from the captured image transmission section 131 is not used for the detection process. For example, the captured image transmitted from the captured image transmission section 131 may be stored in a storage section included in the external device, or may be displayed on a display section.

The image processing section 121 and the switch determination section 122 included in the processing section 120 correspond to the image processing section 221 and the switch determination section 222 included in the external device 200 illustrated in FIG. 2. The process performed by the image processing section 121 and the process performed by the switch determination section 122 are the same as described above, and detailed description thereof is omitted.

The frame rate control section 123 corresponds to the processing section 120 included in the capsule endoscope 100 illustrated in FIG. 2. Specifically, the frame rate control section 123 controls the capture FR based on the determination result (switch instruction) of the switch determination section 122.

According to the configuration illustrated in FIG. 3, the endoscope apparatus 400 can perform the process that detects the halfway position of the small intestine based on the captured image. Therefore, it is possible to reduce a delay until the frame rate is switched to a high frame rate after the captured image has been acquired, as compared with the example illustrated in FIG. 2, and further reduce the possibility that part of the large intestine is not captured.

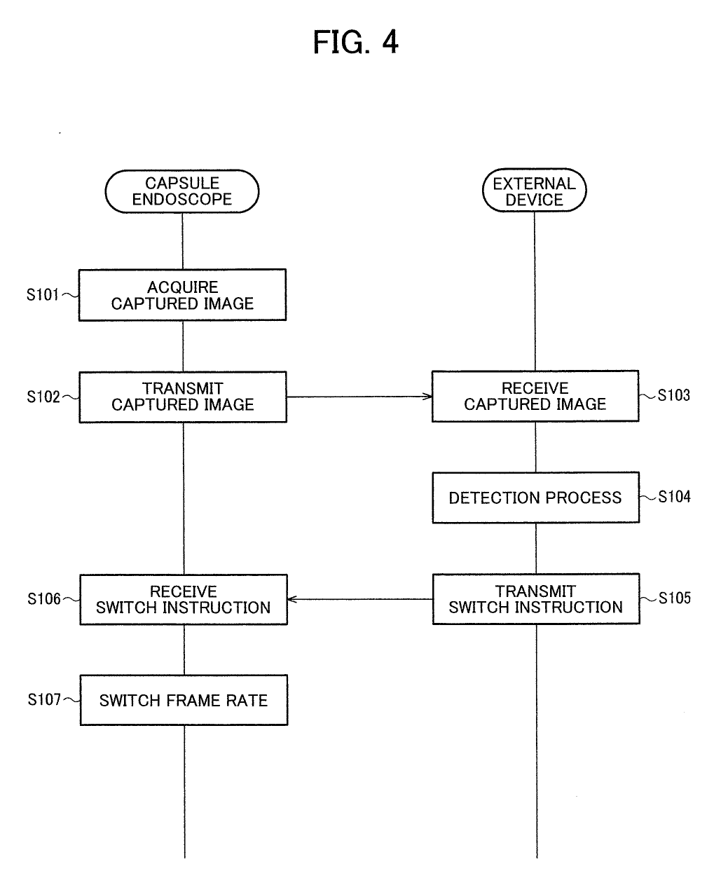

FIG. 4 is a flowchart illustrating the flow of the process according to the first embodiment. The capsule endoscope 100 captures an image (captured image) (S101). The communication section 130 (first communication section) included in the capsule endoscope 100 transmits the captured image to the external device 200 (S102), and the communication section 230 (second communication section) included in the external device 200 receives the captured image (S103).

The processing section 220 (second processing section) included in the external device 200 performs the detection process that detects the halfway position of the small intestine based on the acquired captured image (S104). For example, the processing section 220 performs the detection process that detects the villus distribution. A specific method is described later in connection with the second and third embodiments.

When it has been determined that it is necessary to switch the capture FR as a result of the detection process, the communication section 230 included in the external device 200 transmits the switch instruction (S105), and the communication section 130 included in the capsule endoscope 100 receives the switch instruction (S106). The processing section 120 included in the capsule endoscope 100 switches the capture FR of the imaging section 110 based on the received switch instruction (S107).

According to the first embodiment, the second processing section 220 (switch determination section 222 in a narrow sense) detects a feature quantity of the small intestine that changes from the stomach toward the large intestine from the captured images, and outputs the mode switch instruction based on the detection result.

This makes it possible to appropriately detect the halfway position (given halfway area) of the small intestine. Therefore, it is unnecessary to detect a situation (e.g., the boundary between the small intestine and the large intestine) that is difficult to detect by image processing, and it is possible to perform the determination process (detection process) with high accuracy. Note that the term “feature quantity” used herein refers to a quantity that can be detected from an image (captured image), and represents a feature (e.g., color, texture, gradient, or contour (edge)), or a feature that can be detected by utilizing such a feature.

The second processing section 220 may detect information about the villus distribution from the captured images as the feature of the small intestine that changes from the stomach toward the large intestine, and output the mode switch instruction based on the detection result.

This makes it possible to detect the halfway position of the small intestine using the villus distribution. As described above, no villus is observed in an area from the mouth to the stomach, and no villus is observed in the large intestine. A large amount of villi are observed in the small intestine at a position near the stomach, and the amount of villi decreases as the distance from the large intestine decreases. Specifically, the villus distribution can be used as an index for determining whether or not the object is the small intestine, and determining the position within the small intestine with respect to the small intestine. Therefore, the villus distribution can suitably be used to detect the halfway position of the small intestine. Note that the info illation about the villus distribution may be information that represents the degree of the villus distribution (e.g., the villus score described later in connection with the third embodiment). The information about the villus distribution may be information that represents whether or not each image acquired in time series is a villus image (as described later in connection with the second embodiment), or may be information that represents the number of villus images within a given interval (as described later with reference to FIG. 11).

As described above with reference to FIG. 3, the first embodiment may also be applied to the endoscope apparatus (capsule endoscope) 400 that includes the imaging section 110 that captures the small intestine and the large intestine to acquire a plurality of captured images in time series, and the processing section 120 that controls whether to cause the imaging section 110 to operate in a first mode or a second mode, the first mode being a mode in which the imaging section 110 captures an image at a first frame rate, and the second mode being a mode in which the imaging section 110 captures an image at a second frame rate that is at least higher than the first frame rate, the endoscope apparatus 400 switching the imaging section 110 from the first mode to the second mode at the halfway position of the small intestine based on the captured images, and causing the imaging section 110 to operate in the second mode from the halfway position of the small intestine, and also operate in the second mode in the large intestine.

According to this configuration, the endoscope apparatus 400 can implement the process that switches the mode (capture FR) of the imaging section 110, and the process that detects the halfway position of the small intestine for switching the mode of the imaging section 110. In this case, the processing section 120 included in the endoscope apparatus 400 detects information about the villus distribution from the captured images as the feature quantity of the small intestine that changes from the stomach toward the large intestine, and switches the imaging section 110 from the first mode to the second mode at the halfway position of the small intestine based on the detection result.

For example, the processing section 120 included in the endoscope apparatus 400 may switch the imaging section 110 from the first mode to the second mode when it has been determined that the villus distribution has decreased in a state in which the imaging section 110 operates in the first mode. The details of the method that determines whether or not the villus distribution has decreased are described later in connection with the second and third embodiments.

The second and third embodiments are described later taking an example in which the processing section 220 (second processing section) included in the external device 200 performs the process that detects the halfway position of the small intestine (i.e., villus distribution determination process in a narrow sense). When the method according to the first embodiment is applied to the endoscope apparatus 400 illustrated in FIG. 3, the process that detects the halfway position of the small intestine is performed by the processing section 120 included in the endoscope apparatus 400. Specifically, the process that may be performed by the processing section 220 (second processing section) included in the external device 200 may be performed by the processing section 120 included in the endoscope apparatus 400 illustrated in FIG. 3.

The endoscope system, the endoscope apparatus, and the like according to the first embodiment may include a processor and a memory. The processor may implement the function of each section by means of individual hardware, or may implement the function of each section by means of integrated hardware, for example. For example, the processor may include hardware, and the hardware may include at least one of a circuit that processes a digital signal and a circuit that processes an analog signal. For example, the processor may include one or more circuit devices (e.g., IC), and one or more circuit elements (e.g., resistor or capacitor) that are mounted on a circuit board. The processor may be a central processing unit (CPU), for example. Note that the processor is not limited to a CPU. Various other processors such as a graphics processing unit (GPU) or a digital signal processor (DSP) may also be used. The processor may be a hardware circuit that includes an ASIC. The processor may include an amplifier circuit, a filter circuit, and the like that process an analog signal. The memory may be a semiconductor memory (e.g., SRAM or DRAM), a register, a magnetic storage device (e.g., hard disk drive), or an optical storage device (e.g., optical disk device). For example, the memory stores a computer-readable instruction, and each section of the endoscope system and the endoscope apparatus is implemented by causing the processor to execute the instruction. The instruction may be an instruction included in an instruction set that is included in a program, or may be an instruction that causes a hardware circuit included in the processor to operate.

### 3. Second Embodiment

The configuration of the endoscope system or the endoscope apparatus according to the second embodiment is the same as described above in connection with the first embodiment. The configuration illustrated in FIG. 2 or 3 may be used in connection with the second embodiment. The same elements as those described above in connection with the first embodiment are indicated by the same reference signs (symbols), and description thereof is appropriately omitted. The following description focuses on the differences from the first embodiment.

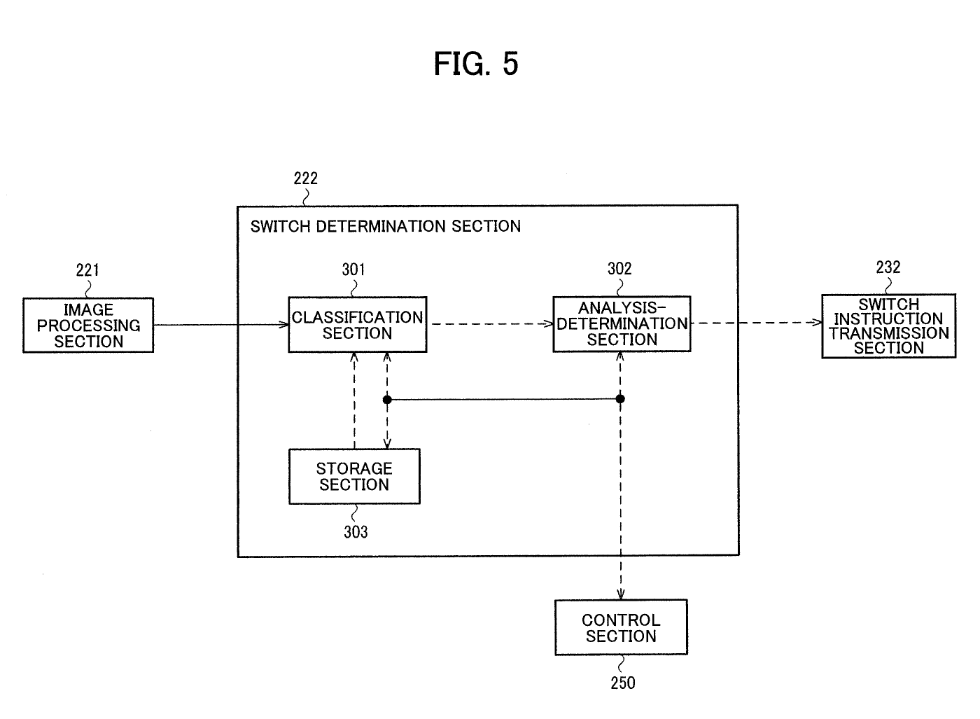

FIG. 5 illustrates an example of the configuration of the switch determination section 222 according to the second embodiment. The switch determination section 222 includes a classification section 301, an analysis-determination section 302, and a storage section 303. The image processing section 221 is connected to the switch instruction transmission section 232 through the classification section 301 and the analysis-determination section 302. The storage section 303 is connected to the classification section 301. The control section 250 is bidirectionally connected to the classification section 301, the analysis-determination section 302, and the storage section 303.

Although FIG. 5 illustrates the configuration of the switch determination section 222 included in the external device 200 illustrated in FIG. 2, the switch determination section 122 included in the endoscope apparatus (capsule endoscope) 400 illustrated in FIG. 3 is also configured the same manner as illustrated in FIG. 5.

In the second embodiment, the villus distribution feature is analyzed based on the results of a learning-classification process that utilizes at least one feature quantity among a color-related feature quantity, a gradient-related feature quantity, and a texture-related feature quantity with respect to the captured image using a known image recognition technique, to detect the given halfway area of the small intestine.

The second embodiment utilizes an image recognition algorithm referred to as “bag-of-features (BoF)” that is independent of the position of the object. The BoF method was developed by applying the bag-of-words method (text retrieval method) to an image recognition process, and includes a learning process and a classification process.

The learning process selects a plurality of learning images. In the second embodiment in which at least two classification items (classification images) including “villus” and “other” are set, an image in which the villus distribution density is high (e.g., an image that includes a large number of villus structures) is determined to be a “villus” learning image, and an image in which the villus distribution density is not high (e.g., an image that includes no villus structure, or an image that includes a small number of villus structures) is determined to be an “other” learning image.

Note that the classification items are not limited thereto. For example, the classification item “villus” may be further classified into classification items “amount of villi is large”, “amount of villi is somewhat large”, “amount of villi is small”, “no villus is observed”, and the like corresponding to the image, and a learning image corresponding thereto may be selected.

A plurality of small sample areas are extracted from the learning image, a feature quantity vector is calculated by a feature quantity extraction process, and a clustering process is performed to select an observation reference referred to as “visual word (VW)”. The second embodiment utilizes a known K-means clustering method. A feature quantity vector is calculated from each small area sequentially extracted from each learning image in the spatial direction, and the distance with respect to the VW is calculated. A vote is cast for the VW for which the distance is a minimum. The voting process is performed on all of the small areas of the learning image to generate a BoF histogram that corresponds to the learning image. The BoF histogram is thus generated in the same number as the number of learning images. A learning classifier that classifies images is generated using the BoF histograms and BoF vectors having the BoF histograms as components. The second embodiment utilizes a learning classifier algorithm referred to as “support vector machine (SVM)”.

A specific learning process is described below. Note that the switch determination section 222 according to the second embodiment of the invention stores the learning results. Therefore, the learning process may be performed by the switch determination section 222, or the learning process may be performed by another block included in the external device 200, or may be performed by another device, and the switch determination section 222 may acquire the results of the learning process.

The learning image is an image for which the relationship between the image and the villus distribution is known in advance. For example, an image obtained in advance by capturing a patient other than the diagnosis target patient is used as the learning image. The plurality of learning images need not be time-series images.

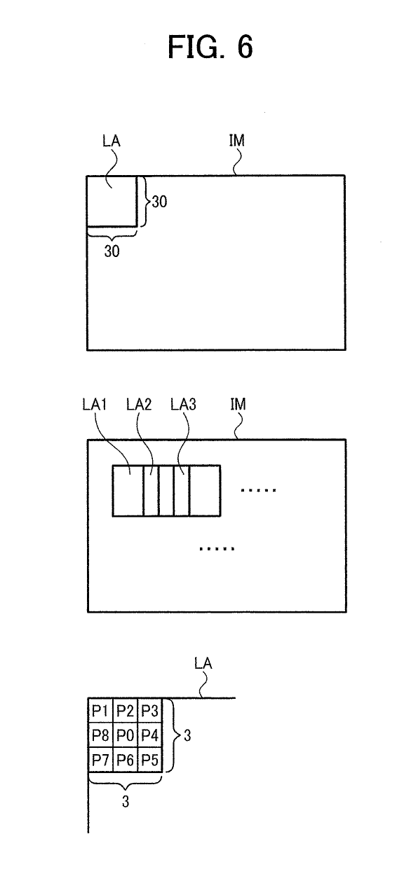

As illustrated in FIG. 6, a plurality of local areas LA having a given size are set to an image IM (one learning image). Specifically, a plurality of local areas LA1, LA2, LA3, . . . are set so as to overlap each other. For example, when the image IM includes 300×300 pixels, each local area is set to include 30×30 pixels. Note that the size of the local area may be changed corresponding to the size of the image IM.

A locally binary pattern (LBP) is applied to the image of each local area LA, for example. The LBP value is calculated from 3×3 pixels that include each pixel of the local area LA as the center pixel. The center pixel of the 3×3 pixels is referred to as P0, and the pixels situated around the center pixel are referred to as P1 to P9. The pixel value of each of the pixels P1 to P9 is compared with the pixel value of the pixel P0. A value “1” is assigned to a pixel that has a pixel value equal to or larger than that of the pixel P0, and a value “0” is assigned to a pixel that has a pixel value smaller than that of the pixel P0. These values (bits) are arranged in order from P1 to P9 to obtain an 8-bit value.

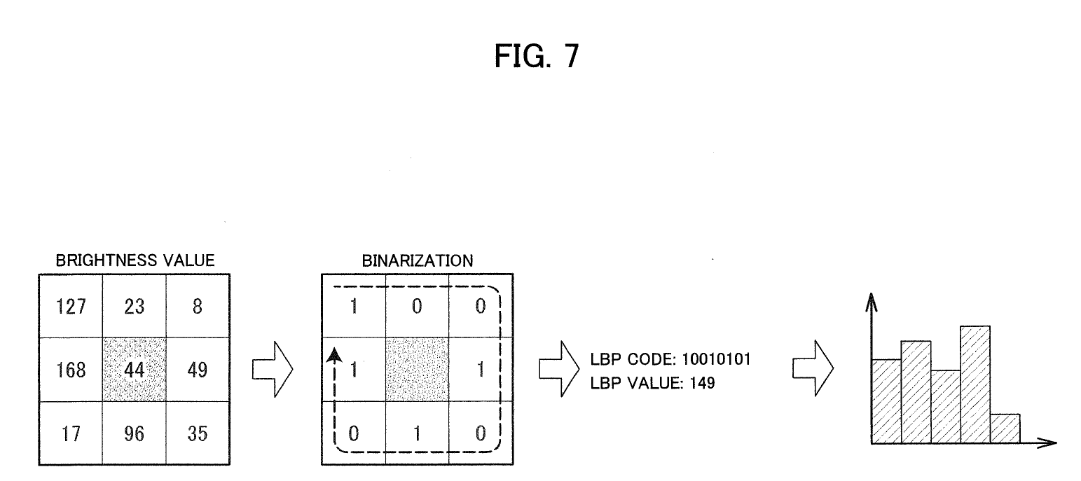

The above process is performed on each pixel of the local area LA to obtain 900 (=30×30) LBP values per local area LA. The 900 LBP values are classified into values “0” to “255”. The numbers of values classified into these values are counted to obtain a 256-dimensional local feature histogram with respect to each local area LA. A normalization process is performed on a block basis to obtain a 256-dimensional feature vector (local feature quantity). The local feature quantity is calculated corresponding to each of the local areas LA1, LA2, . . . to generate local feature quantities in the same number as the number of local areas. FIG. 7 illustrates the local feature quantity calculation process described above.

The process that calculates the local feature quantity from the local area is performed on a plurality of images, and a number of vectors are stored as the local feature quantities. For example, when the number of learning images is 100, and 100 local areas are set to each image, 10,000 local feature quantities are acquired.

A clustering process is performed on the stored local feature quantities using a K-means clustering method to extract a representative vector. The representative vector corresponds to the VW (see above). The K-means clustering method sets the number of classes to k, sets k representative vectors to an initial state, classifies the feature vectors into k classes, calculates the average position of each class, moves the representative vectors, and classifies the feature vectors into k classes. This process is repeated to determine the final classes. For example, the number k of representative vectors (VW) is set to 100.

A representative vector for which the Euclidean distance between the local feature quantity and the representative vector is a minimum is determined from the 100 representative vectors. This process is performed on each image on a local area basis. A number (1 to 100) is assigned to the 100 representative vectors, and the number of local areas for which the Euclidean distance with respect to each representative vector is a minimum is counted to generate a 100-dimensional histogram. The histogram is generated on a learning image basis. The histogram is considered to be a 100-dimensional vector, and determined to be the BoF (bag-of-features) feature vector of the image.

One BoF feature vector is acquired from one learning image by performing the above process. The BoF feature vectors in the same number as the number of learning images is linked to a correct answer label (e.g., “villus” or “other”) to generate a learning data set.

A learning process is performed using the learning data set by means of a support vector machine (SVM), for example. The SVM is a learner that determines the label separating plane (e.g., a plane that separates the feature vectors “villus” and “other”) in the feature vector space from the learning data set. For example, a linear separation process is performed in the feature vector space to determine the separating plane. Alternatively, a linear separation process may be performed in a higher-dimensional vector space to determine a non-linear separating plane with respect to the dimensions of the feature vector. In the second embodiment, the results of the learning process are stored in the storage section 303.

The classification process sequentially inputs the classification target captured images, calculates the local feature quantity from each small area sequentially extracted from the captured image in the spatial direction, and calculates the distance with respect to the VW. A vote is cast for the VW for which the distance is a minimum. The voting process is performed on all of the small areas of the captured image to calculate one BoF feature vector (BoF histogram) from the captured image.

The classification process is performed using the SVM classifier obtained by the learning process and the BoF feature vector acquired from the captured image, and the classification results are output. Specifically, the classification section 301 generates the BoF histogram using the captured image output from the image processing section 221, and extracts and compares the SVM classifier output from the storage section 303, the BoF histogram generated from the learning image, and the information about the BoF feature vector to generate a classification index that represents that the captured image belongs to “villus” or “other”, under control of the control section 250. Specifically, the classification section 301 calculates a villus score that represents the probability that the captured image belongs to “villus”, and an other score that represents the probability that the captured image belongs to “other”. The classification section 301 transmits the classification index of the captured image to the analysis-determination section 302.

In the second embodiment, the feature quantity vector is calculated using at least one feature quantity among a color-related feature quantity, a gradient-related feature quantity, and a texture-related feature quantity with respect to the captured image.

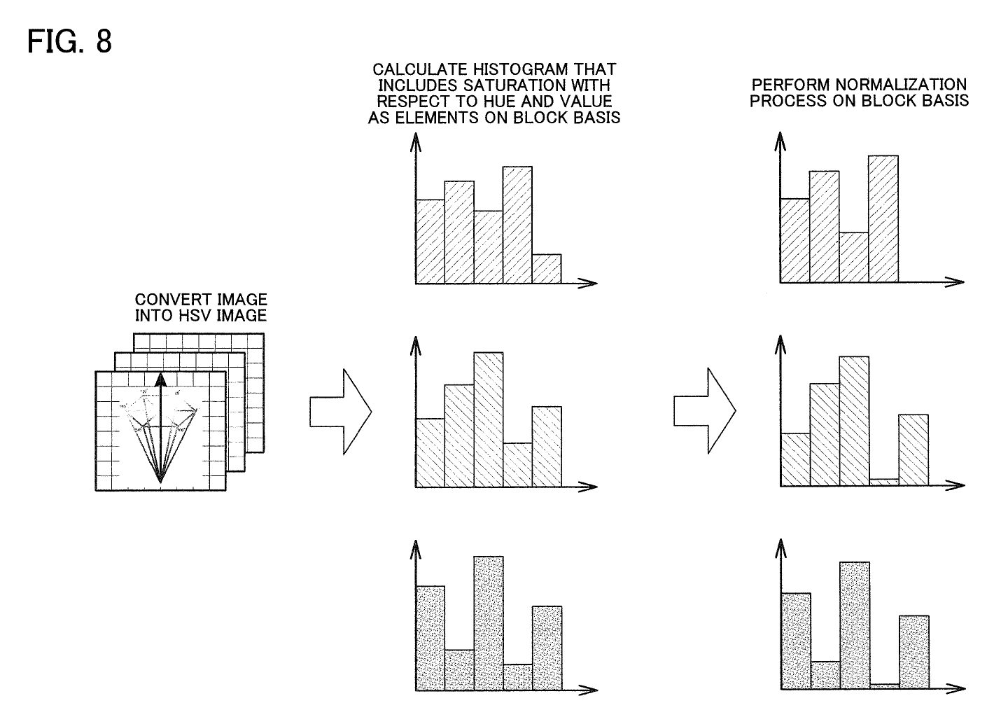

The gradient-related feature quantity is the LBP (see above), for example. The color-related feature quantity may be a hue-saturation-value (HSV) (see FIG. 8). The HSV is a color space that consists of a hue component, a saturation component, and a value component. FIG. 8 illustrates an example of a local feature quantity calculation process that uses the HSV. The HSV color space is divided into a plurality of areas respectively in the hue direction, the saturation direction, and the value direction. The image is converted into the HSV color system on a pixel basis. The HSV image is divided into a plurality of blocks, and a histogram that includes saturation with respect to hue and value as elements is calculated on a block basis. The above process is performed after moving the block to generate histograms in the same number as the number of blocks included in one image. A normalization process is performed on a block basis to generate an HSV feature quantity vector.

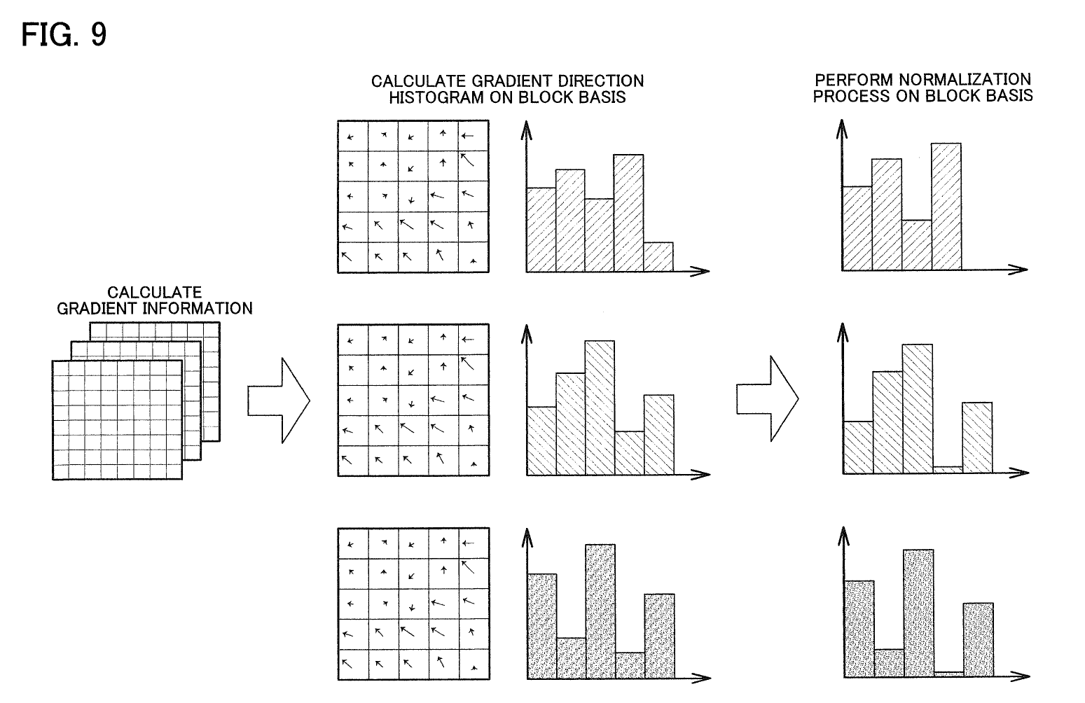

The texture-related feature quantity may be the histogram of oriented gradients (HOG) illustrated in FIG. 9. FIG. 9 illustrates an example of a local feature quantity calculation process that uses the HOG. The local area of the image is divided into a plurality of blocks, and brightness gradient information (e.g., gradient direction and weight) is calculated on a pixel basis to calculate a brightness gradient histogram on a block basis. The above process is performed after moving the block to generate histograms in the same number as the number of blocks included in one image. A normalization process is performed on a block basis to generate an HOG feature quantity vector.

Although an example in which the learning process and the classification process are performed using the LBP feature quantity, the HSV feature quantity, and the HOG feature quantity, has been described above, the configuration is not limited thereto. For example, the learning process and the classification process are performed using an arbitrary gradient-related feature quantity, an arbitrary color-related feature quantity, and an arbitrary texture-related feature quantity, as required.

A feature vector in which a plurality of local feature quantities are combined, may be generated. The color, the gradient, and the texture may be combined using an early fusion method that combines the color, the gradient, and the texture in an early stage of the process, or a late fusion method that combines the color, the gradient, and the texture in a late stage of the process.

For example, the early fusion method represents a 3×3-pixel pattern in each local area using a combination of a uniform LBP (ULBP) feature quantity (texture feature quantity) and the HSV color feature of the center pixel. For example, the HSV color space is divided into 12 sections in the hue direction, and divided into 3 sections in the saturation direction, and the achromatic value is divided into 4 sections. In this case, the feature quantity is a 40-dimensional feature quantity. Since the ULBP feature quantity is a 10-dimensional feature quantity, a 400 (=40×10)-dimensional feature quantity is generated by the early fusion method.

For example, the late fusion method determines a joint histogram obtained by arranging the BoF histogram and the LBP histogram of the HSV color feature quantity to be the feature vector of the image. Alternatively, the late fusion method performs a learning process on the color, the texture, or a combination of the color and the texture (by means of early fusion or late fusion) using a classifier (e.g., SVM) (described later), adds up the classification scores obtained by the classification process, and performs a threshold value determination process. A learner-classifier with higher accuracy can be obtained by combining the above methods.

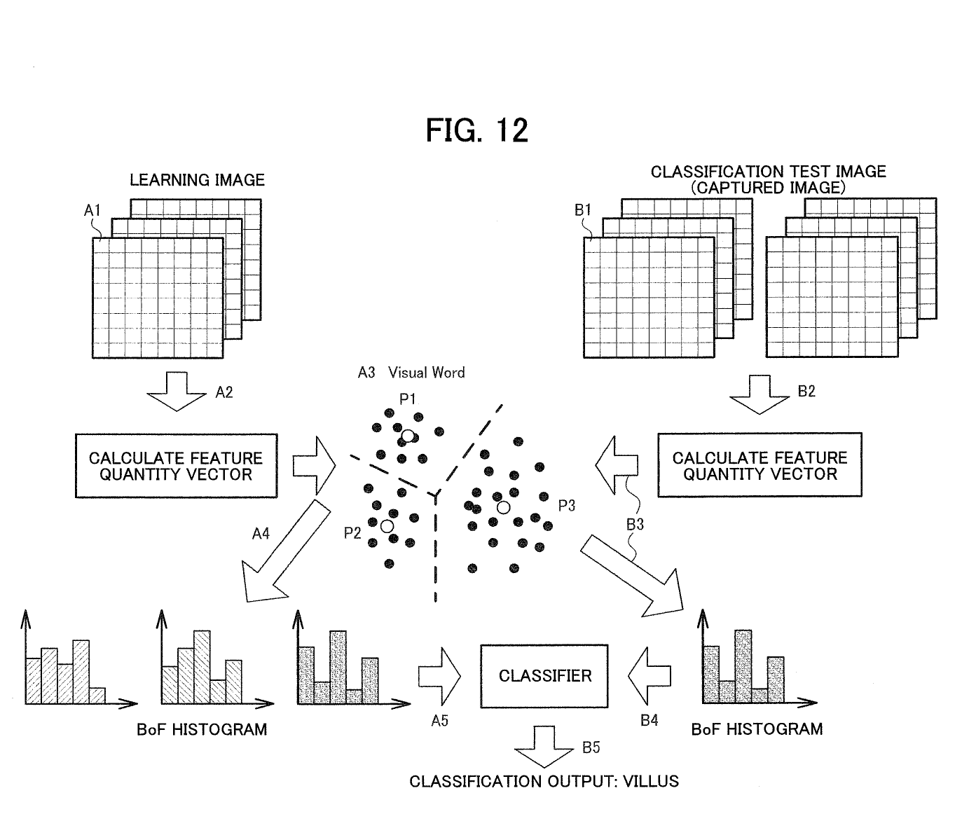

FIG. 12 illustrates the flow of the learning process and the classification process. In FIG. 12, the left side illustrates the learning stage. Specifically, the learning image is divided into a plurality of local areas, and the local feature quantity is calculated (A1 and A2). This process is performed on a plurality of learning images to calculate a number of local feature quantities, and the VW is set (A3). One BoF feature vector (BoF histogram) is calculated from one learning image based on the distance between the VW and the local feature quantity (A4). The correct answer data (tag “villus” or “other”) has been assigned to each learning image, and the classifier (e.g., SVM) is generated from the BoF feature vector and the correct answer data (A5).

During the classification process, the captured image (test image) is divided into a plurality of local areas, and the local feature quantity is calculated (B1 and B2). One BoF feature vector (BoF histogram) is calculated from one learning image based on the distance between the VW set in the step A3 and the local feature quantity (B3). The captured image is classified as one of a plurality of classification items using the BoF feature vector and the classifier generated in the step A5 (B4 and B5).

The analysis-determination section 302 calculates the villus distribution using the number of images classified as “villus” or “other” within the interval that includes a plurality of images captured in time series under control of the control section 250. FIG. 11 illustrates an example of a distribution measurement process (“villus” or “other”). N (wherein N is an integer equal to or larger than 2) images among a plurality of images captured in time series are set to be a distribution measurement interval. The number of images classified as “villus” is counted within the distribution measurement interval. When the number of images classified as “villus” is larger than a given threshold value th1, the distribution measurement interval is determined to be the villus interval. The number of images classified as “other” is counted within the distribution measurement interval. When the number of images classified as “other” is larger than a given threshold value th2, the distribution measurement interval is determined to be the other interval. When the determination process has been performed on the distribution measurement interval, a new interval is set by shifting the position by n (n≧1) images in the time-series direction, and the determination process is performed on the new interval that includes N images. The above process is repeated to determine whether each interval is the villus interval or the other interval.

It is anatomically defined that villi are distributed only in the small intestine. The villus distribution density is high in the first half area of the small intestine, and decreases to some extent in the ileum area. The second embodiment utilizes a capsule endoscope that is used to make a diagnosis with respect to the large intestine. After the capsule endoscope 100 has been swallowed, an image is captured at a low frame rate. When it has been determined by the above determination process that the villus interval has occurred for the first time after the capsule endoscope 100 has been swallowed, it is determined that the capsule endoscope 100 has entered the small intestine. In this case, an image is continuously captured at a low frame rate. When it has been determined that the other interval has occurred after the villus interval, it is determined that the capsule endoscope 100 has reached the given halfway area of the small intestine, and the capture frame rate is switched from a low frame rate to a high frame rate.

As described above, an image is captured at a low frame rate after the capsule endoscope 100 has been swallowed, and whether the determination target interval that includes a given number of images captured in time series is the villus interval or the other interval is determined using the number of images classified as “villus” or “other”. The given halfway area of the small intestine in which the villus distribution density decreases is determined, and the capture frame rate is switched from a low frame rate to a high frame rate. The latter half of the small intestine and the large intestine are captured at a high frame rate. This makes it possible to prevent a situation in which part of the large intestine is not captured (i.e., a correct diagnosis may not made with respect to the large intestine), and reduce the power consumption of the capsule endoscope 100.

Although an example in which the capture frame rate is switched from a low frame rate to a high frame rate when the given halfway area of the small intestine has been determined, and an image is then continuously captured at a high frame rate, has been described above, the configuration is not limited thereto.

A modification of the second embodiment is described below. It is anatomically defined that the villus distribution density is high in the first half area of the small intestine, and decreases to some extent in the ileum area. However, this definition is based on the assumption that the villus distribution density in the first half area of the small intestine is higher than that in the ileum area on average, and the first half area of the small intestine may include an area in which the villus distribution density is very high, and an area in which the villus distribution density is relatively low, depending on the patient. The ileum area may also include an area in which the villus distribution density is relatively low, and an area in which the villus distribution density is slightly high. Specifically, while the villus distribution density in the first half area of the small intestine is higher than that in the ileum area on average, the villus distribution density may vary in the first half area of the small intestine (i.e., the first half area of the small intestine may include an area in which the villus distribution density is relatively low). Since the capsule endoscope 100 moves due to a physical motion inside the body, the capsule endoscope 100 moves forward and backward (i.e., does not necessarily move forward). For example, the capsule endoscope 100 may enter the small intestine from the stomach, and then return to the stomach.

Specifically, since it is also determined by the above determination process that the other interval has occurred after the villus interval when the villus distribution density decreases in the first half area of the small intestine, or when the capsule endoscope 100 has entered the small intestine from the stomach, and then returned to the stomach, an image is continuously captured at a high frame rate after the capture frame rate has been switched from a low frame rate to a high frame rate until the capsule endoscope 100 is discharged from the body.

Therefore, power consumption increases, and the battery may become empty before the capsule endoscope 100 is discharged from the body. In order to deal with the above problem, the modification proposes the following method. Specifically, the capture frame rate is immediately switched from a low frame rate to a high frame rate when the other interval has occurred, and is switched from a high frame rate to a low frame rate when the villus interval has occurred again. That is, the capture frame rate is switched from a low frame rate to a high frame rate, or switched from a high frame rate to a low frame rate, each time the villus interval or the other interval has occurred.

This makes it possible to appropriately set the capture frame rate to a low frame rate when it is undesirable to continuously capture an image at a high frame rate (see above), and reduce power consumption. Note that the capture frame rate may be set (fixed) to a high frame rate when a given time has elapsed after the capsule endoscope 100 has been swallowed in order to prevent a situation in which an erroneous diagnosis is made, or the interval is erroneously determined to be the villus interval or the other interval. Various other modifications and variations may also be made.

According to the second embodiment, the second processing section 220 outputs the mode switch instruction that instructs to switch from the first mode to the second mode when it has been determined that the villus distribution has decreased in a state in which the imaging section 110 operates in the first mode.

In the example described above, whether or not the villus distribution has decreased is determined based on the number of images that have been determined to be “villus” within the determination interval (N images). In this case, whether or not the number of images that have been determined to be “villus” has decreased may be determined. Alternatively, whether or not the interval is the villus interval or the other interval may be determined using the threshold value determination process, and it may be determined that the villus distribution has decreased when the other interval has occurred after the villus interval (see above).

This makes it possible to switch the mode to the second mode (i.e., high-frame-rate mode) when it has been determined that the villus distribution has decreased. It is known that the villus distribution in the small intestine decreases on average as the distance from the large intestine decreases. Specifically, a situation in which the villus distribution has decreased to a certain extent means that the capsule endoscope has approached the large intestine to a certain extent with respect to the start position of the small intestine. It is possible to appropriately reduce power consumption, and prevent a situation in which part of the large intestine is not captured, by utilizing a decrease in villus distribution as a trigger for switching the frame rate to a high frame rate.

The second processing section 220 may output the mode switch instruction that instructs to cause the imaging section 110 to operate in the first mode when it has been determined that the villus distribution has increased.

A situation in which the villus distribution has increased corresponds to a situation in which the object (capture target) has changed from the stomach in which the villus distribution is not observed, to the small intestine in which the villus distribution is observed. Specifically, it is possible to capture the object that follows the start position of the small intestine in the first mode by causing the imaging section 110 to operate in the first mode using an increase in villus distribution as a trigger. Note that it is indispensable to capture an image using the imaging section 110 in order to determine the villus distribution from the captured image. Specifically, it is unlikely that the imaging section 110 is not operated even before the villus distribution increases. For example, the imaging section 110 may be set to a mode in which the imaging section 110 captures an image at a 0th frame rate that is lower than the first frame rate before the villus distribution increases.

Alternatively, the imaging section 110 may be necessarily operated in the first mode after the capsule endoscope has been swallowed by the user until the villus distribution decreases. In this case, an increase in villus distribution does not serve as a trigger for switching the capture FR.

The second processing section 220 may classify the plurality of captured images that have been captured by the imaging section 110 into a plurality of classification images that include at least a first classification image and a second classification image, the first classification image being an image for which it has been determined that the villus distribution is large, and the second classification image being an image for which it has been determined that the villus distribution is small, calculate the villus distribution based on the frequency of at least one classification image among the plurality of the classification images, and output the mode switch instruction that instructs to switch from the first mode to the second mode based on the villus distribution.

This makes it possible to classify each captured image as one of a plurality of classification images (classification items), and calculate the villus distribution based on the classification results. Since the classification item according to the second embodiment represents the degree of villus distribution (see above), the classification results that represent the classification items into which the captured images have been classified can be used as information that represents the degree of villus distribution included in each captured image.

Specifically, the second processing section 220 may acquire classification information calculated by a learning process, and classify the plurality of captured images that have been captured by the imaging section 110 into the plurality of classification images based on a feature quantity and the classification information, the feature quantity being calculated from each of the plurality of captured images that have been captured by the imaging section 110.

This makes it possible to perform the classification process based on the learning process. Although an example in which the BoF feature vector is used as the learning feature quantity, and the SVM is used as the classifier, has been described above, the classifier may be generated using another method.

The second processing section 220 may set a determination interval that includes N (wherein N is an integer equal to or larger than 2) captured images acquired in time series, determine that the villus distribution is small when the number of captured images among the N captured images that have been classified as the first classification image is equal to or smaller than th1 (wherein th1 is a positive integer equal to or smaller than N), or when the number of captured images among the N captured images that have been classified as the second classification image is equal to or larger than th2 (wherein th2 is a positive integer equal to or smaller than N), and output the mode switch instruction that instructs to switch from the first mode to the second mode.

This makes it possible to detect the halfway position of the small intestine, and output the mode switch instruction using the number of images classified as “villus” or the number of images classified as “other” within a given interval (see FIG. 11). When the number of classification items (classification images) is equal to or larger than 3, the frequency of an arbitrary classification item may be used, and the frequencies of two or more classification items may be used in combination. Although an example in which the classification item under which the determination interval falls is determined, has been described above, the configuration is not limited thereto. a time-series change in the number (or the ratio) of images may be determined.

The second processing section 220 may output the mode switch instruction that instructs to switch from the second mode to the first mode when it has been detei lined that the villus distribution has increased in a state in which the imaging section 110 operates in the second mode.

This makes it possible to return the mode to the first mode (low-frame-rate mode) after the mode has been switched to the second mode when it has been determined that it was inappropriate to switch the mode to the second mode, and reduce unnecessary electricity consumption. It is determined that it was inappropriate to switch the mode to the second mode when the capsule endoscope has returned to the stomach from the small intestine (see above), for example.

The first processing section 120 may cause the imaging section 110 to operate at the first frame rate in the first mode, and cause the imaging section 110 to operate at the second frame rate or a third frame rate in the second mode, the third frame rate being higher than the second frame rate. In this case, the second processing section 220 outputs a frame rate switch instruction that instructs to cause the imaging section 110 to operate at the second frame rate or the third frame rate in a state in which the imaging section 110 operates in the second mode, and the first processing section 120 causes the imaging section 110 to operate at the second frame rate or the third frame rate based on the frame rate switch instruction.

This makes it possible to switch between a plurality of capture FR in the second mode (high-frame-rate mode). When a given object is captured at the third frame rate, it is possible to further reduce the possibility that the given object is not captured.

The second processing section 220 may output the frame rate switch instruction that instructs to cause the imaging section 110 to operate at the second frame rate or the third frame rate based on the villus distribution or motion information about the capsule endoscope 100 in a state in which the imaging section 110 operates in the second mode.

This makes it possible to switch between the high frame rate (second frame rate) and the super-high frame rate (third frame rate) based on the villus distribution or the motion information. For example, when the villus distribution is used, two threshold values may be provided with respect to the ratio of images determined to be “villus” within the determination interval. The frame rate may be switched from the first frame rate to the second frame rate (i.e., the mode may be switched from the first mode to the second mode) when the ratio has become less than a threshold value T1, and may be switched from the second frame rate to the third frame rate (in the second mode) when the ratio has become less than a threshold value T2 (<T1). Since it is considered that the villus distribution is sufficiently small at a position situated sufficiently near the large intestine, it is possible to increase the possibility that the large intestine (object of interest) can be captured at the third frame rate, and while reducing power consumption by reducing the time in which the imaging section 110 operates at the third frame rate as much as possible. In this case, since an area around the start position of the large intestine may not be captured when only the threshold value T2, it is effective to set the threshold value T1.

When the motion information is used, the frame rate may be set to the third frame rate when the amount of motion is large. Since the moving distance of the capsule endoscope 100 increases when the amount of motion is large, and the possibility that the object is not captured increases. It is possible to reduce the possibility that the object is not captured, by setting the frame rate to the third frame rate when the amount of motion is large.

### 4. Third Embodiment

The configuration of the endoscope system or the endoscope apparatus according to the third embodiment is the same as described above in connection with the first embodiment. The configuration illustrated in FIG. 2 or 3 may be used in connection with the third embodiment. The same elements as those described above in connection with the first embodiment are indicated by the same reference signs (symbols), and description thereof is appropriately omitted. The following description focuses on the differences from the first embodiment.

In the second embodiment, each captured image is classified as “villus” or “other”. However, one captured image normally includes a plurality of classification items. For example, one captured image may include both a “villus” area and an “other” area.

In the second embodiment, the SVM classifier output from the storage section 303, and the information about the BoF histogram from the captured image (and the BoF feature vector that uses the BoF histogram as a component), are extracted and compared to generate the classification index that represents that the captured image belongs to “villus” or “other”. When the captured image includes both a “villus” area and an “other” area, the “villus” SVM score and the “other” SVM score are calculated with respect to the “villus” area and the “other” area, and compared to provide the captured image with the classification index that corresponds to the classification item with a higher SVM score. When the captured image includes areas that correspond to three or more classification items, the captured image is provided with the classification index that corresponds to the classification item with the highest SVM score.

Specifically, when the classification method described above in connection with the second embodiment is used, the captured image is provided with the classification item with the highest SVM score, but may include an area that corresponds to another classification item.

The villus distribution differs depending on the observation target (patient) (see above). For example, the amount of villi may be very large or very small over the entire small intestine depending on the patient. In this case, a determination error may occur when the captured image is classified using the classification item with the highest SVM score, and the interval is determined to be the villus interval or the other interval based on the classification results.

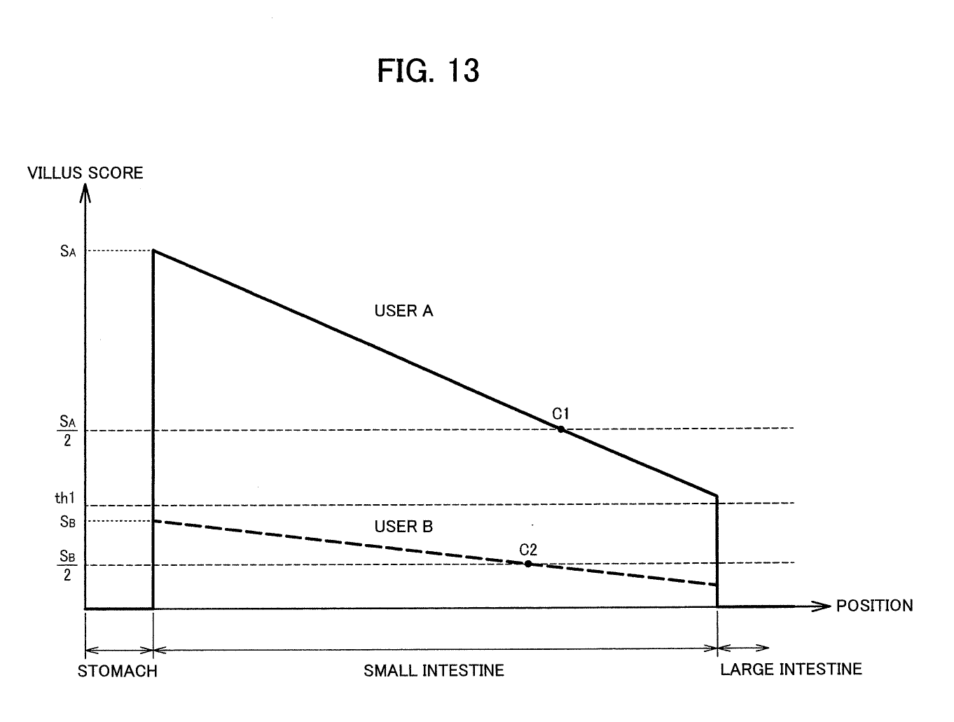

FIG. 13 is a schematic view illustrating a specific example. In FIG. 13, the horizontal axis indicates the position inside the body, and the vertical axis indicates the amount of villi (i.e., the villus score that corresponds to the amount of villi). The left side along the horizontal axis indicates the mouth side, and the right side along the horizontal axis indicates the anus side. Although FIG. 13 illustrates an example in which the villus score decreases linearly and monotonously for convenience, the villus score decreases non-linearly in the actual situation, and may not necessarily decrease monotonously. For example, the target image is classified as “villus” when the villus score has exceeded th1.

In the example illustrated in FIG. 13, since the user A has a large amount of villi (i.e., the villus score is high), the villus score does not become less than th1. Therefore, it is difficult to detect the halfway position of the small intestine, and appropriately switch the capture FR. Since the amount of villi is small in the large intestine, an instruction that instructs to switch the frame rate to a high frame rate may be issued when the capsule endoscope has entered the large intestine. In this case, however, it is likely that an area around the start position of the large intestine is not captured. On the other hand, since the user B has a small amount of villi (i.e., the villus score is low), the villus score does not exceed th1. Therefore, it is difficult to appropriately switch the capture FR since the captured image is not classified as “villus” even when the capsule endoscope has entered the small intestine.

Although FIG. 13 illustrates an example in which it is difficult to switch the capture FR, it is undesirable that an individual variation be large even in a situation in which the capture FR can be switched at the halfway position of the small intestine. Specifically, since the position within the small intestine at which the frame rate is switched to a high frame rate (e.g., a movement ratio when the start position of the small intestine is 0%, and the end position of the small intestine is 100%) changes corresponding to the villus score, the frame rate may be switched to a high frame rate at a position within the small intestine situated on the side of the mouth, or may be switched to, a high frame rate at a position within the small intestine situated on the side of the anus, depending on the user. In this case, it may be difficult to effectively reduce power consumption if the frame rate is switched to a high frame rate at a position within the small intestine situated excessively on the side of the mouth, and part of the large intestine may not be captured if the frame rate is switched to a high frame rate at a position within the small intestine situated excessively on the side of the anus. Specifically, it is desirable that the frame rate be switched to a high frame rate independently of an individual variation in the amount of villi at a position that ensures that power consumption can be effectively reduced while effectively preventing a situation in which part of the large intestine is not captured.

In the third embodiment, the given halfway area of the small intestine is determined by the based on the change rate of the SVM score with respect to a plurality of intervals that include a plurality of images using the “villus” SVM score or the “other” SVM score of the captured image regardless of the item for which the SVM score becomes a maximum.

For example, the “villus” SVM score of each captured image captured after the capsule endoscope has been swallowed by the patient is added up and averaged on an interval basis. When the change rate of the “villus” average SVM score with respect to each interval has exceeded a given threshold value for the first time, the interval is determined to correspond to the small intestine area. When the change rate of the “villus” average SVM score with respect to one interval has become less than the given threshold value, the interval is determined to correspond to the given halfway area of the small intestine, and the frame rate is switched from a low frame rate to a high frame rate.

This makes it possible to switch the frame rate to a high frame rate at an appropriate position (timing) by absorbing an individual variation with respect to the user. For example, a position at which the villus score has decreased by 50% with respect to the villus score at the start position of the small intestine (i.e., a position at which the villus score has increased for the first time) may be determined to be the halfway position (given area) of the small intestine. In the example illustrated in FIG. 13, the switch position with respect to the user A is the position C1 at which the villus score reaches half of the score SA at the start position, and the switch position with respect to the user B is the position C2 at which the villus score reaches half of the score SB at the start position. It is possible to adaptively control the capture frame rate corresponding to the features of the villus distribution of each patient, and reduce the occurrence of a determination error, by thus switching the frame rate based on the change rate of the “villus” or “other” SVM score of “Other” or “Villus”. with respect to a plurality of intervals.

Note that it is also possible to take account of a situation in which the capsule endoscope 100 that has entered the small intestine returns to the stomach. Specifically, when it has been determined that the capsule endoscope 100 has returned to the stomach after the frame rate has been switched to a high frame rate, the frame rate may be returned to a low frame rate. In this case, the villus score (or the other score) is also used instead of the classification results (“villus” or “other”). Specifically, when the “villus” average SVM score (or the change rate of the average SVM score with respect to a given reference) with respect to the interval has exceeded the given threshold value after the frame rate has been switched to a high frame rate, the frame rate may be returned to a low frame rate.

According to the third embodiment, the second processing section 220 calculates the villus score that represents the degree of the villus distribution with respect to each of the plurality of captured images that have been captured by the imaging section 110, and outputs the mode switch instruction that instructs to switch from the first mode to the second mode based on a time-series change in the villus score.

The villus score may be an SVM score that is calculated using an SVM, for example. Since a classifier that is acquired by an ordinary learning process calculates a score with respect to each classification item during classification, such a score may be used when a classifier other than an SVM is used. The villus score may be a score that corresponds to “villus”. Note that the villus score is not limited thereto. For example, a score that corresponds to “other” may also be used. Specifically, since a score that corresponds to “other” is an index that represents that the amount of villi is small, a situation in which the score that corresponds to “other” is low (high) is synonymous with a situation in which the score that corresponds to “villus” is high (low).

This makes it possible to detect the halfway position of the small intestine taking account of an individual variation with respect to the user. When each captured image is classified using an item for which the score becomes a maximum (see the second embodiment), it is difficult to appropriately switch the mode (frame rate) depending on the user (see FIG. 13). On the other hand, it is possible to implement an appropriate detection process independently of the absolute amount of villi of each user by utilizing a time-series change in the villus score.

The first to third embodiments according to the invention and the modifications thereof have been described above. Note that the invention is not limited to the first and to third embodiments and the modifications thereof. Various modifications and variations may be made without departing from the scope of the invention. A plurality of elements among the elements described above in connection with the first to third embodiments and the modifications thereof may be appropriately combined to implement various other configurations. For example, an arbitrary element may be omitted from the elements described above in connection with the first to third embodiments and the modifications thereof. Some of the elements described above in connection with the first to third embodiments and the modifications thereof may be appropriately combined. Any term cited with a different term having a broader meaning or the same meaning at least once in the specification and the drawings can be replaced by the different term in any place in the specification and the drawings. Accordingly, various modifications and applications are possible without materially departing from the novel teachings and advantages of the invention.

What is claimed is:

1. An endoscope system comprising:
a capsule endoscope; and
an external device,
the capsule endoscope comprising:
an imaging section that captures a small intestine and a large intestine to acquire a plurality of captured images in time series;
a first processor that comprises hardware, and controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate; and
a first communication section that transmits the captured images to the external device, and
the external device comprising:
a second processor that comprises hardware, and outputs a mode switch instruction based on the captured images, the mode switch instruction instructing to switch from the first mode to the second mode at a halfway position of the small intestine; and
a second communication section that transmits the mode switch instruction to the first communication section,
wherein the first processor switches the imaging section from the first mode to the second mode at the halfway position of the small intestine based on the mode switch instruction, and causes the imaging section to operate in the second mode from the halfway position of the small intestine, and also operate in the second mode in the large intestine.

2. The endoscope system as defined in claim 1,
wherein the second processor detects a feature quantity of the small intestine that changes from a stomach toward the large intestine from the captured images, and outputs the mode switch instruction based on a detection result.

3. The endoscope system as defined in claim 2,
wherein the second processor detects information about a villus distribution from the captured images as the feature quantity of the small intestine that changes from the stomach toward the large intestine, and outputs the mode switch instruction based on the detection result.

4. The endoscope system as defined in claim 3,
wherein the second processor outputs the mode switch instruction that instructs to switch from the first mode to the second mode when it has been determined that the villus distribution has decreased in a state in which the imaging section operates in the first mode.

5. The endoscope system as defined in claim 3,
wherein the second processor outputs the mode switch instruction that instructs to cause the imaging section to operate in the first mode when it has been determined that the villus distribution has increased.

6. The endoscope system as defined in claim 3,
wherein the second processor classifies the plurality of captured images that have been captured by the imaging section into a plurality of classification images that comprise at least a first classification image and a second classification image, the first classification image being an image for which it has been determined that the villus distribution is large, and the second classification image being an image for which it has been determined that the villus distribution is small, calculates the villus distribution based on a frequency of at least one classification image among the plurality of classification images, and outputs the mode switch instruction that instructs to switch from the first mode to the second mode based on the villus distribution.

7. The endoscope system as defined in claim 6,
wherein the second processor acquires classification information calculated by a learning process, and classifies the plurality of captured images that have been captured by the imaging section into the plurality of classification images based on the feature quantity calculated from each of the plurality of captured images, and the classification information.

8. The endoscope system as defined in claim 7,
wherein the second processor sets a determination interval that includes N (wherein N is an integer equal to or larger than 2) captured images acquired in time series, determines that the villus distribution is small when a number of captured images among the N captured images that have been classified as the first classification image is equal to or smaller than th1 (wherein th1 is a positive integer equal to or smaller than N), or when a number of captured images among the N captured images that have been classified as the second classification image is equal to or larger than th2 (wherein th2 is a positive integer equal to or smaller than N), and outputs the mode switch instruction that instructs to switch from the first mode to the second mode.

9. The endoscope system as defined in claim 3,
wherein the second processor calculates a villus score that represents a degree of the villus distribution with respect to each of the plurality of captured images that have been captured by the imaging section, and outputs the mode switch instruction that instructs to switch from the first mode to the second mode based on a time-series change in the villus score.

10. The endoscope system as defined in claim 3,
wherein the second processor outputs the mode switch instruction that instructs to switch from the second mode to the first mode when it has been determined that the villus distribution has increased in a state in which the imaging section operates in the second mode.

11. The endoscope system as defined in claim 3,
wherein the first processor causes the imaging section to operate at the first frame rate in the first mode, and causes the imaging section to operate at the second frame rate or a third frame rate in the second mode, the third frame rate being higher than the second frame rate,
the second processor outputs a frame rate switch instruction that instructs to cause the imaging section to operate at the second frame rate or the third frame rate in a state in which the imaging section operates in the second mode, and
the first processor causes the imaging section to operate at the second frame rate or the third frame rate based on the frame rate switch instruction.

12. The endoscope system as defined in claim 11,
wherein the second processor outputs the frame rate switch instruction that instructs to cause the imaging section to operate at the second frame rate or the third frame rate based on the villus distribution or motion information about the capsule endoscope in a state in which the imaging section operates in the second mode.

13. An endoscope apparatus comprising:
an imaging section that captures a small intestine and a large intestine to acquire a plurality of captured images in time series; and
a processor that comprises hardware, and controls whether to cause the imaging section to operate in a first mode or a second mode, the first mode being a mode in which the imaging section captures an image at a first frame rate, and the second mode being a mode in which the imaging section captures an image at a second frame rate that is at least higher than the first frame rate,
the endoscope apparatus switching the imaging section from the first mode to the second mode at a halfway position of the small intestine based on the captured images, and causing the imaging section to operate in the second mode from the halfway position of the small intestine, and also operate in the second mode in the large intestine.

14. The endoscope apparatus as defined in claim 13,
wherein the processor detects information about a villus distribution from the captured images as a feature quantity of the small intestine that changes from a stomach toward the large intestine, and switches the imaging section from the first mode to the second mode at the halfway position of the small intestine based on a detection result.

15. The endoscope apparatus as defined in claim 14,
wherein the processor switches the imaging section from the first mode to the second mode when it has been determined that the villus distribution has decreased in a state in which the imaging section operates in the first mode.

16. A method for controlling an endoscope system comprising:
causing an imaging section to capture a small intestine and a large intestine to acquire a plurality of captured images in time series;
outputting a mode switch instruction based on the captured images, the mode switch instruction instructing to switch from a first mode to a second mode at a halfway position of the small intestine, the first mode being a mode in which the imaging section captures an image at a first frame rate, and the second mode being a mode in which the imaging section captures an image at at least a second frame rate that is higher than the first frame rate; and
switching the imaging section from the first mode to the second mode at the halfway position of the small intestine based on the mode switch instruction, and causing the imaging section to operate in the second mode from the halfway position of the small intestine, and also operate in the second mode in the large intestine.
